(19) United States
(12) Patent Application Publication
(10) Pub. No.: US 2003/0035786 A1
(43) Pub. Date: Feb. 20, 2003

(54) Biological tissue adhesives, articles, and methods

(75) Inventors: Marc Hendriks, Brunssum (NL); Michel Verhoeven, Maastricht (NL); Patrick Cahalan, Windham, NH (US); Vincent Larik, Kerkrade (NL)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/132,516

(22) Filed: Apr. 26, 2002

Related U.S. Application Data
(62) Division of application No. 09/433,564, filed on Nov. 4, 1999.

(51) Int. Cl. A61K 38/39; C08H 1/00

(52) U.S. Cl. 424/78.17; 514/21; 606/214; 514/2; 527/204

(57) ABSTRACT

Biological tissue adhesives can be in the form of a gel that is applied to biological tissue as a “glue” or supported on a backing or substrate to form an article such as a self-sticking patch or pad. Adhesive articles can include such biological tissue adhesives or be functionalized to directly adhere to biological tissue without the biological adhesive.

### BACKGROUND OF THE INVENTION

Traditionally, mechanical methods have been used to seal wounds in biological tissue. These include sutures, staples, tapes, and bandages. These may or may not be bioabsorbable. More recently, medical adhesives or biological glues (also referred to as tissue sealants or tissue adhesives) have been used for both internal and external applications, such as tissue adhesion, hemostasis, and sealing of air and body fluid leaks in surgery.

Most epimysial and epineurial electrodes make use of some sort of synthetic pad or patch for reliable fixation to the tissue of choice. For example, one such patch electrode includes a defibrillation lead featuring a polytetrafluoroethylene (TEFLON) felt pad in which three parallel stainless steel defibrillation electrodes are mounted. Examples of such products include Medtronic Lead Model 13004 and are disclosed in U.S. Pat. No. 5,527,358. The primary purpose of the pad is to reliably fix the defibrillation lead to the atrium, and to protect the atrial wall for electrical damage. Typically, such pads are sutured in place; however, it would be desirable to use medical adhesives or biological glues to hold them in place.

Currently available tissue adhesives include cyanoacrylate adhesives, fibrin glues (U.S. Pat. Nos. 5,883,078; 5,464,471; 5,407,671; 4,909,251; 4,414,976; 4,377,572; 4,362,567; and 4,298,598), and gelatin/resorcinol/formaldehyde adhesives. Many currently available adhesives have several disadvantages. These include, for example, high cost, toxicity, the need for elaborate measures for biological safety to prevent transfer of infections as a result of the use of constituents derived from human blood, and/or the formation of cured polymers that are more stiff than natural tissues.

New approaches toward the development of a safe, effective tissue adhesive have recently been identified. For example, a rapidly curable biological glue composed of two food additives, i.e., poly(L-glutamic acid) and gelatin, has been disclosed that is chemically crosslinked by a water-soluble carbodiimide (CDI) when in contact with the biological tissue (i.e., the reaction occurs in situ). The resultant cured adhesive includes residual carbodiimide, as disclosed in Otani et al., J. Biomed. Mater. Research, 31, 157-166 (1996). This is undesirable because such carbodiimides are known to elicit a cytotoxic response when in contact with biological tissue.

Another recently disclosed approach involves the use of the grafting of sulfur-containing cysteine residues onto gelatin chains. These cysteine residues are a very good precursor to the formation of di-sulfur bridges U.S. Pat. No. 5,412,076), which are natural protein crosslinks that can be obtained in the presence of a mild oxidizer, such as iodine. As such, the gelatin can be crosslinked to tissue and act as an adhesive. This approach seems more reasonable from a safety perspective, although actual adhesion is dependent upon adding a mild oxidizer.

U.S. Pat. Nos. 5,900,245 and 5,552,452 disclose tissue adhesive systems which form an adhesive bond after exposure of the adhesive to photoactivating radiation.

U.S. Pat. No. 5,549,904 discloses a biological adhesive composition utilizing tissue transglutaminase in an aqueous carrier. The tissue transglutaminase is used in a catalytic amount to promote adhesion between tissue surfaces by catalyzing the reaction between glutaminyl residues and amine donors of the tissue and/or the enzyme. The carrier contains a divalent metal ion such as calcium to promote the reaction.

U.S. Pat. Nos. 5,936,035 and 5,817,303 both disclose adhesive systems based upon utilization of proteinaceous polymers, naturally occuring or produced by recombinant techniques, having functionalities for crosslinking to provide adherent tissue adhesives and sealants. U.S. Pat. No. 5,936,035 particularly discloses the utilization of polyethyleneglycol crosslinking reagents containing activated carboxyl groups capable of reacting with tissue amine groups. U.S. Pat. No. 5,817,303 particularly discloses the utilization of di-aldehyde crosslinking reagents (such as glutaraldehyde), and di-isocyanate crosslinking reagents (such as polymethylene diisocyanate). Other crosslinking reagents such as acid anhydrides and di-amino compounds are also disclosed.

Matsuda et al., J. Biomed. Mater. Research, 45, 20-27 (1999), disclosed the utilization of glutaraldehyde to make a gelatin film bioadhesive. The adhesion of the gelatin film is based on formation of covalent bonds through formation of a Schiff base with amino groups of tissues.

In the above disclosures many new methods to generate bioadhesive glues or articles have been described. All these approaches do have their own strengths, but some more often than not do also have weaknesses.

The adhesive systems that are based on photoactivation as disclosed in U.S. Pat. No. 5,900,245 and U.S. Pat. No. 5,552,452 are very elegant, but still actual adhesion is dependent on coming in with an additional means (e.g., reactants), in this case the light source. For that reason the methods described in U.S. Pat. Nos. 5,936,035 and 5,817,303 are more preferable, as these systems allow for in situ adhesion without any additional means. However, these systems have some weaknesses as well. Glutaraldehyde has been shown to induce cytotoxicity when applied in crosslinking of tissue or other collagenous materials. Speer and coworkers found that glutaraldehyde concentrations as low as 3 ppm completely inhibited H3-thymidine uptake by fibroblasts, a measure for cytotoxicity (Speer et al., J Biomed. Mater. Res., 23,1355-1365 (1989)). While the approach disclosed by Matsuda et al. is based upon introduction of dangling aldehyde groups into the gelatin material, and as such it can be claimed that no free glutaraldehyde molecules are available, which should lead to reduced cytotoxic potential, the formed Schiff base is of a reversible nature, and can only be permanently stabilized through reductive amination.

Several di-isocyanates are available and have been studied in the reactions with amino acids and proteins (Wold, Methods Enzymol., 25, 623-651 (1972)). In a similar manner as glutaraldehyde, the isocyanate group reacts with the amine groups of tissue resulting in crosslinking between tissue and the adhesive system. The main disadvantage of the isocyanate is its susceptibility to hydrolysis. As a consequence, the use of di-isocyanates will yield formation of pendant molecules containing amine groups. This has been suggested to cause secondary cytotoxicity, i.e., release of toxic products as a result of enzymatic actions (Van Luyn et al., Mat. Res. Soc. Symp. Proc., 252, 167-174 (1992)). The presence of the di-isocyanate hydrolysis product, 1,6-diaminohexane (DAH), within the material will impact its biocompabitility also, due to direct leakage of the toxic DAH from the adhesive system (Yano et al., Jpn. J. Ind. Health, 23, 537-543 (1981)).

Active esters, also referred to as activated carboxyl groups, are very susceptible to hydrolysis, and thus become easily deactivated (Grabarek et al., Anal. Biochem., 185, 131-135 (1990)). Also, the hydrolysis induced release of the ‘activators’ may lead to increased inflammatory responses at the application site.

### SUMMARY OF THE INVENTION

There is a continuing need for biological tissue adhesives that have sufficient biocompatibility, thereby resulting in low cytotoxicity and reduced inflammatory response, such that there is no interference in the normal healing process. Such adhesives desirably have substantial bond strength for either internal or external tissues and good mechanical strength after cure. Preferably, they should form adhesive bonds in an aqueous environment without the addition of other reactants. This requires that desirably these adhesives have enhanced stability towards hydrolysis. This means that the functional group responsible for the adhesive activity desirably becomes less easily deactivated in an aqueous environment.

The present invention provides biological tissue adhesives, articles (e.g., self-sticking patches or pads), and methods of adhering. The adhesives include functional groups that are capable of covalently bonding to biological tissue, whether it be internal or external tissue, under aqueous conditions. Preferred such functional groups include isocyanates, vinylsulfones, and activated esters, with vinylsulfones being the most preferred.

In one embodiment, the present invention provides an adhesive article (e.g., a self-sticking pad) that includes a solid support (preferably, a water-insoluble solid support) having covalently bound (i.e., covalently bonded) functional groups pendant therefrom which are reactive with biological tissue when in an aqueous environment (which can come from added water or the water present in biological tissue) to cause adhesion of the support to the tissue. Preferably, the water-insoluble support includes collagen and the functional groups are selected from the group of isocyanates, vinylsulfones, activated esters, and mixtures thereof. More preferably, the functional groups are selected from the group of isocyanates, vinylsulfones, and mixtures thereof. Most preferably, the functional groups are vinylsulfones.

In a particularly preferred embodiment, the present invention provides a biological adhesive article that includes a solid support of collagen and covalently bound functional groups pendant therefrom. The functional groups are selected such that they are reactive with biological tissue when in an aqueous environment sufficient to cause adhesion of the support to the tissue through the formation of covalent bonds. Preferaby, the functional groups are selected from the group of isocyanates, vinylsulfones, activated esters, and mixtures thereof. Most preferably, the functional groups are vinylsulfones.

It is believed that adhesion to tissue is caused by forming covalent bonds between the functional groups and the biological tissue when in an aqueous environment. It is further believed that the amine and/or thiol groups of the tissue form a part of these bonds. However, all embodiments of the present invention are not necessarily so limited.

The present invention also provides a method of attaching an article (e.g., a self-sticking pad) to biological tissue. The method includes providing a biological adhesive article that includes a solid support (preferably comprising a collagen matrix) having covalently bound functional groups pendant therefrom which are selected such that they are reactive with biological tissue when in an aqueous environment, and contacting the biological adhesive article to the biological tissue to cause adhesion of the support to the tissue. Preferably, adhesion occurs through the formation of covalent bonds between the functional groups and the biological tissue. Preferably, in this method, the biological adhesive article is contacted with water prior to contacting it to the biological tissue.

Another embodiment of the invention involves a method of preparing a self-sticking pad. The method includes providing a solid support (preferably, one that includes collagen), and functionalizing the solid support with functional groups selected such that they are reactive with biological tissue when in an aqueous environment to cause adhesion of the support to the tissue. Typically, the functional groups are provided by chemically modifying the solid support to form pendant functional groups directly bonded to the solid support.

In another embodiment, the present invention provides a biological adhesive comprising a stable complex of one or more crosslinkable biomolecules having vinylsulfone functional groups. Such groups, when under aqueous conditions, are capable of bonding to biological tissue. As used herein, a “stable” complex is one that retains its crosslinkable activity during storage, preferably when stored in a substantially dry environment. This includes preformed adhesives, as opposed to compositions that are prepared in situ (i.e., when in contact with the tissue), such that the preformed adhesives have the ability to bond (preferably, set up covalent bonds) to biological tissue.

The present invention also provides a method of sealing a wound. The method includes contacting the wound with a biological adhesive comprising a stable complex of one or more crosslinkable biomolecules comprising vinylsulfone functional groups. Preferably, the vinylsulfone functional groups form covalent bonds with the biological tissue (particularly the amine and/or thiol groups of the biological tissue) when in an aqueous environment.

A method of forming a biological adhesive is also provided. In one embodiment, the method includes combining gelatin with one or more crosslinkable biomolecules comprising vinylsulfone functional groups. In this embodiment, there may or may not be covalent interaction between the gelatin and the biomolecules. In another embodiment, the method includes chemically modifying the gelatin with vinylsulfone functional groups.

In another embodiment, the present invention provides a biological adhesive comprising a stable complex of one or more crosslinkable biomolecules having free amine groups, a portion of which are blocked, and functional groups, which, under aqueous conditions, are capable of bonding to biological tissue. The present invention also provides a method of sealing a wound using this adhesive.

Methods of making these biological adhesives are also provided. In one embodiment, gelatin is combined with one or more crosslinkable biomolecules comprising free amine groups, a portion of which are blocked, and functional groups, which, under aqueous conditions, are capable of bonding to biological tissue. In another embodiment, gelatin is chemically modified with fundtional groups, which, under aqueous conditions, are capable of bonding to biological tissue, wherein the gelatin comprises free amine groups, a portion of which are blocked.

### BRIEF DESCRIPTION OF THE DRAWINGS

FIG. 1. General reaction mechanism for vinylsulfone functionalization of collagen.

FIG. 2. General reaction mechanism for diisocyanate functionalization of collagen.

FIG. 3. General reaction mechanism for active ester functionalization of collagen after majority of free amine groups are blocked.

FIG. 4. FT-IR spectra of collagen sheets treated with NHS-PEG-VS in different solvents.

FIG. 5. FT-IR spectra of collagen sheets treated with HMDI in different solvents. Reference spectrum of HMDI is included as well.

FIG. 6. A top view of the testing system used to measure the adhesion or bonding strength of a vinylsulfone self-sticking collagen sheet to porcine heart tissue. The insert illustrates the positioning of the collagen sheet in between the two pieces of heart tissue.

FIG. 7. A graphical representation of the results of the bonding strength test of a vinylsulfone self-sticking collagen sheet to porcine heart tissue.

### DETAILED DESCRIPTION OF THE INVENTION

The adhesives of the present invention can be in the form of a gel that is applied to biological tissue as a “glue” or supported on a substrate (i.e., support) to form an article such as a self-sticking patch or pad. Thus, the biological adhesives of the present invention can be used alone or they can be coated on or bonded to a substrate. In such embodiments, a biological adhesive includes a stable complex of one or more crosslinkable biomolecules comprising functional groups, which, under aqueous conditions, are capable of bonding to biological tissue (e.g., wound sites and organs such as heart, colon, pancreas, etc.). A stable complex is one that retains its crosslinkable activity during storage. Preferably, storage conditions include a substantially dry environment.

In yet another embodiment, the substrate (i.e., support) can include functional groups, which, under aqueous conditions, are capable of bonding to biological tissue. These functional groups are typically an integral part of the support formed without the use of a biological adhesive as described above.

In all embodiments, preferred functional groups include vinylsulfones, isocyanates, activated esters, and mixtures thereof. For certain embodiments, the functional groups are preferably vinylsulfones. When contacted with water (which can be added or simply present in the biological tissue), these functional groups adhere to biological tissue. This adhesion is believed to result from the formation of covalent bonds to reactive groups, such as amine groups and sulhydryl groups, on biological tissue.

The biological adhesives and adhesive articles of the present invention can be further modified to include bioactive molecules, including therapeutic agents.

Support

The support is preferably substantially water-insoluble, although this is not a necessary requirement. That is, little if any of the support is solubilized in water over a wide range of temperatures and pressures, particularly room temperature and body temperature. The support is preferably solid at room temperature (i.e., about 20° C. to about 30° C.).

The support can be in a variety of forms with a variety of materials. Examples of the types of materials include fabrics (e.g., felt), sponge, polymeric sheeting (e.g., films or membranes) of a variety of natural and synthetic polymers. The supports can include combinations of materials, such as a laminated material consisting of a sponge on one side and a film on the other side. More layers are possible, as well. Preferably, the support is in the form of a thin sheet of material. More preferably, the support is in the form of a combination of sponge and film (bi-layered), or even film-sponge-film (triple layered), as these provide more body to the covering.

The support can include synthetically produced biodegradable polymers, such as the following polymer families: poly(amino acids); lactide-glycolide copolymers; polyanhydrides; polyhydroxybutyrates; poly(ortho ester)s; and poly(phospho ester)s. Preferably, the synthetic biodegradable polymer includes polylactic acid, polyglycolic acid, polydioxanone, poly(ε-)caprolactan, poly(α-)malic acid, poly(β-)malic acid, polyhydroxybutyric acid, tyrosine-derived polyiminocarbonates, tyrosine-derived polycarbonates, and tyrosine-derived polyarylates.

While less preferential, the support can also include biostable, biocompatible materials, such as polyurethane, silicone rubber, polyesters (e.g., DACRON), fluoropolymers such as polytetrafluoroethylene (e.g., TEFLON), polyvinylidenefluoride, and TEFZEL, polyimides, PEBAX, and others. Other possible materials for the support include metals and ceramics, such as stainless steel, titanium, tantalum, and others.

Preferred materials of the support include collagen as well as other proteinaceous materials, whether or not crosslinked or otherwise chemically or physically modified, such as gelatin, keratin, elastin, fibrin, and albumin. Other materials include glycosaminoglycans and polysaccharides, whether or not crosslinked or otherwise chemically or physically modified, such as dermatan sulfates, chondroitin sulfates, heparin, heparan sulfates, hyaluronic acid, cyclodextrins, starch, dextrans, dextran sulfates, chitin, and chitosan.

Collagen is a preferred material for use in the support. The collagen can be part of a collagen-based material including whole tissue (i.e., tissue containing collagen and noncollagenous substances or cells), only the collagen matrix without the noncollagenous substances, or, more preferably, reconstituted and purified collagen. In certain preferred embodiments, the above materials, such as the biostable materials, can form a fibrous network enclosed within a collagen matrix.

Collagen is a naturally occurring protein featuring good biocompatibility. It is the major structural component of vertebrates, forming extracellular fibers or networks in practically every tissue of the body, including skin, bone, cartilage, and blood vessels. In medical devices, collagen provides a more physiological, isotropic environment that has been shown to promote the growth and function of different cell types, facilitating the rapid overgrowth of host tissue after implantation.

Basically, three types of collagen-based materials can be identified, based on the differences in the purity and integrity of the collagen fiber bundle network initially present in the material. The first type includes whole tissue including noncollagenous substances or cells. As a result of using whole tissue, the naturally occurring composition and the native strength and structure of the collagen fiber bundle network are preserved. Whole tissue xenografts have been used in construction of heart valve prostheses, and also in vascular prostheses. However, the presence of soluble proteins, glycoproteins, glycosaminoglycans, and cellular components in such whole tissue xenografts may induce an immunological response of the host organism to the implant.

The second type of collagen-based material includes only the collagen matrix without the noncollagenous substances. The naturally occurring structure of the collagen fiber bundle network is thus preserved, but the antigenicity of the material is reduced. The fibrous collagen materials obtained by removing the antigenic noncollagenous substances will generally have suitable mechanical properties.

The third type of collagen-based material is reconstituted and purified collagen. Purified collagen is obtained from whole tissue by first dispersing or solubilizing the whole tissue by either mechanical or enzymatic action. The collagen dispersion or solution is then reconstituted by either air drying, lyophilizing, or precipitating out the collagen. A variety of geometrical shapes like sheets, tubes, sponges or fibers can be obtained from the collagen in this way. The resulting materials, however, do not have the mechanical strength of the naturally occurring fibrous collagen structure.

Typically, in order to use collagen-based materials in medical devices, at least a portion of the collagen is crosslinked. Crosslinking of collagen-based materials is used to suppress the antigenicity of the material. In addition, crosslinking is used to improve mechanical properties and enhance resistance to degradation. Crosslinking can be performed by means of physical methods, including, for example, UV irradiation and dehydrothermal crosslinking. Several chemical crosslinking methods for collagen-based materials are known. These methods involve, for example, the reaction of a bifunctional reagent with the amine groups of lysine or hydroxylysine residues on different polypeptide chains or the activation of carboxyl groups of glutamic and aspartic acid residues followed by the reaction with an amine group of another polypeptide chain to give an amide bond.

In a preferential setting, at least a portion of the collagen is crosslinked in order to enhance its biostability; however, the collagen can also be used if it is not crosslinked. It is preferred that after crosslinking sufficient reactive groups remain within the collagen material which can be used to bind the molecular substance (e.g., functional groups or biological adhesive) that is used to give the support its adhesive characteristics.

As stated above an adhesive article (e.g., a self-sticking pad) that includes a support can have either vinylsulfone functional groups, isocyanate functional groups, activated ester functional groups, or combinations thereof. When contacted with water, adhesion of the support to the tissue occurs. The normal moisture in biological tissue in certain situations may be sufficient to initiate adhesion. Soaking of the adhesive article prior to contact with the bodily tissue in a saline solution is preferred, although in certain situations excess wetting might prohibit quick adhesion. This was confirmed by Matsuda et al., J. Biomed. Mater. Research, 45, 20-27 (1999), who disclosed that bonding strength was significantly reduced after full hydration of a gelatin sheet. When the adhesive article is a thin sheet it is preferably applied dry. Spongy material may need to be wet when applied to avoid local dehydration of tissues and consequent tissue damage.

The collagen matrix can also be used to load or couple bioactive molecules. As a result, a material can be produced that actively participates in the host-material interaction, thereby enhancing the acceptance and performance of the material. A wide variety of known bioactive molecules can be used according to the present invention. Examples include, but are not limited to, angiogenic factors, growth factors, antimicrobial agents, antithrombotic agents, anticalcification agents, anti-inflammatory agents, an anti-arrhythmic agent, an analgesic and other therapeutic agents.

Supports described herein can be modified with a biological adhesive as by coating, laminating, bonding, etc. Alternatively, the supports can be chemically functionalized with groups capable of adhering to biological tissue. The latter is preferred for certain embodiments of the present invention. This can be done using a variety of reaction schemes. The following discussion focuses on: (1) the chemical modification of a solid support (collagen) to form pendant functional groups, such as vinylsulfone, isocyanate, or activated ester groups, directly bonded thereto; and (2) gelatin, which is a suspension of hydrolyzed collagen, modified with activated ester groups or vinyl sulfone groups to form a biological glue. These are provided for exemplification purposes only. The invention is not to be limited thereby. With this disclosure, one of skill in the art will be able to apply such chemistries (or other chemistries) to other materials and form biological adhesives or self-sticking pads. For example, the chemistry described for the vinylsulfone and isocyanate functionalized collagen supports can be modified and used to convert gelatin into a biological glue.

Vinylsulfone Functionalized Collagen Support

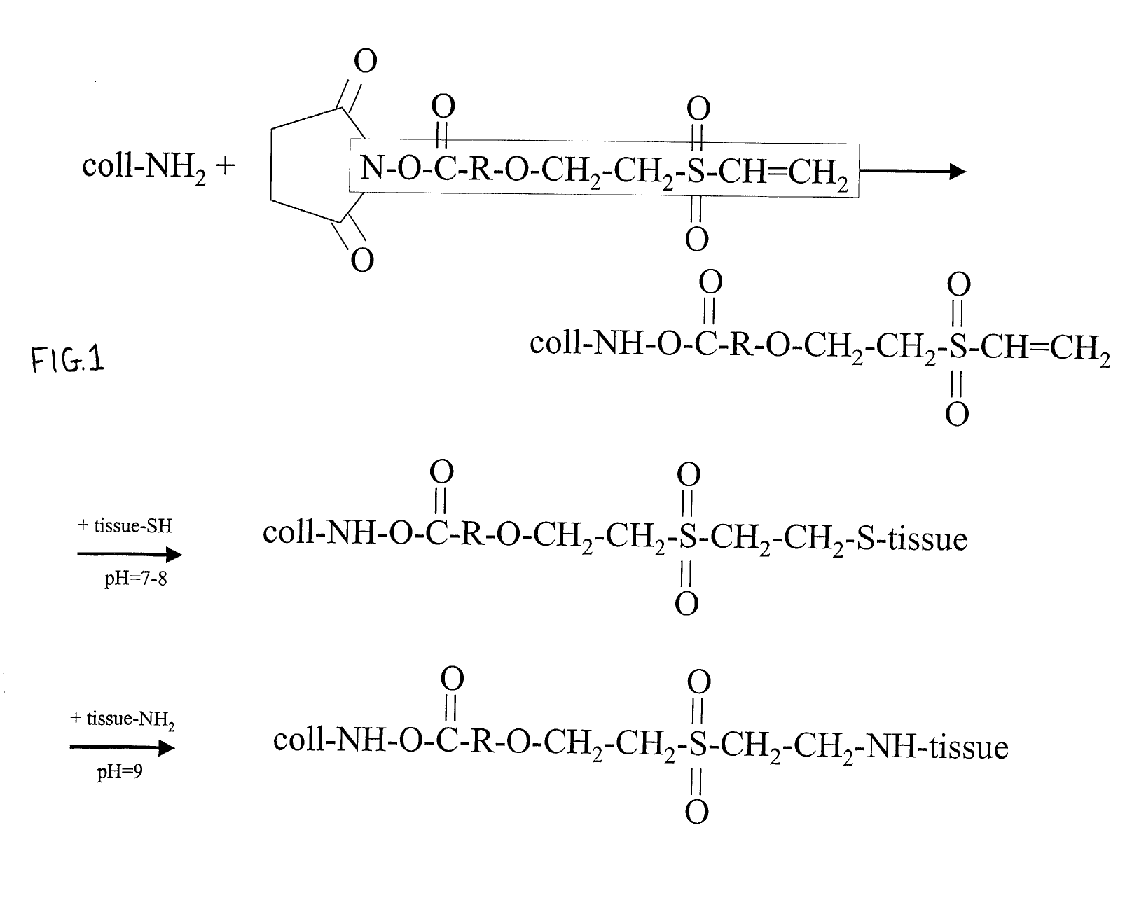

As shown in FIG. 1, in one aspect of the present invention, collagen amine groups (e.g., a (hydroxy)lysine amine group) react with one end of a bifunctional vinylsulfone-containing compound, such that the vinylsulfone (VS) functionality is available for subsequent reactions. Preferably, the one end of the bifunctional vinylsulfone-containing compound is an activated carboxyl group, also referred to as active ester, but various other functional groups capable of reacting with amines may be employed as well, such as aldehydes, isocyanates, acid anhydrides, vinylsulfones, and the like.

More preferably, the activated carboxyl group is an N-hydroxysuccinimide (NHS) activated carboxyl group. NHS-PEG-VS is an especially useful heterofunctional compound. The NHS ester group is highly reactive toward amino groups, but is hydrolytically unstable. Contrarily, the vinylsulfone group is hydrolytically stable. The vinylsulfone (VS) end groups are selective for reaction with sulfhydryl groups around pH 7, while reaction with amino groups proceeds at higher pH.

Thus the NHS-PEG-VS can be used to provide adhesive characteristics to the collagen by first coupling to an amino group by means of the NHS ester, followed by reaction of the dangling VS group with sulfhydryl or amino groups in tissues. The advantage of this system is that the hydrolytic stability of vinylsulfone makes possible a leisurely approach to the second step. In the scope of this invention, this allows for generation of an article with adhesive characteristics (through the available vinylsulfone groups), such as a self-sticking pad, that shows enhanced stability of its adhesive function during normal storage conditions when compared to those methods disclosed by others.

Suitable vinylsulfones are of the formula

NHS—O—C(O)—R—O—CH₂—CH₂—SO₂—HC═CH₂

wherein NHS—O— represents N-hydroxysuccinimide ester and R is a divalent organic linking group, preferably an aliphatic group optionally substituted with oxygen atoms. More preferably, R is an alkylene group (preferably, having 4-12 carbon atoms) or a polyoxyalkylene group (preferably having an approximate MW of 150-5000).

The following list provides a few commercially available examples. NHS-PEG-VS (available with various molecular weights), from Shearwater Polymers, Huntsville, U.S.A.); succinimidyl-(4-vinylsulfonyl)benzoate (SVSB) and 1,6 hexane-bis-vinylsulfone (HBVS), both from Molecular Biosciences, Inc., Boulder, U.S.A.; and 1,3-bis(vinylsulfonyl)propane, 1,4-bis(vinylsulfonyl)butane, 1,4-bis(vinylsulfonylmethyl)benzene, and divinylsulfone, all from Sigma-Aldrich Chemie BV, Zwijndrecht, The Netherlands.

These and/or other commercially available compounds are preferred, but it can be appreciated that other compounds that are appropriate for utilization according to this invention can be synthesized as well.

Of these, the heterofunctional NHS-PEG-VS compound is most preferred, as it allows for steering the reaction by controlling the pH, such that pendant VS functionalities are most effectively introduced.

Other compounds that can be used to convert the carboxylic acid end of the vinylsulfone-containing molecule into an activated ester (besides NHS) include: hydroxybenzotriazole (HOBt), N-hydroxy-5-norbornene-endo-2,3-dicarboximide (HONB), 4-dimethylaminopyridine (DMAP), and the sulfo-derivative of N-hydroxysuccinimide (sulfo-NHS), 1-ethyl-3-(3-dimethylaminopropyl)carbodiimide (EDC), cyanamide, N,N′-dicyclohexylcarbodiimide (DCC), N,N′-diisopropylcarbodiimide (DIC), 1-cyclohexyl-3-(2-morpholinoethyl)carbodiimide metho-p-toluenesulfonate (CMC), 1,1′-carbonyldiimidazole (CDI), N,N′-disuccinimidyl carbonate (DSC), 2-ethoxy-1-ethoxycarbonyl-1,2-dihydroquinoline (EEDQ), 1,2-benzisoxazol-3-yl-diphenyl phosphate (BDP), and N-ethyl-5-phenylisoxazolium-s′-sulfonate (Woodwards Reagent K).

This mechanism of functionalizing a support containing collagen, for example, is exemplified in Example 1.

Isocyanate Functionalized Collagen Support

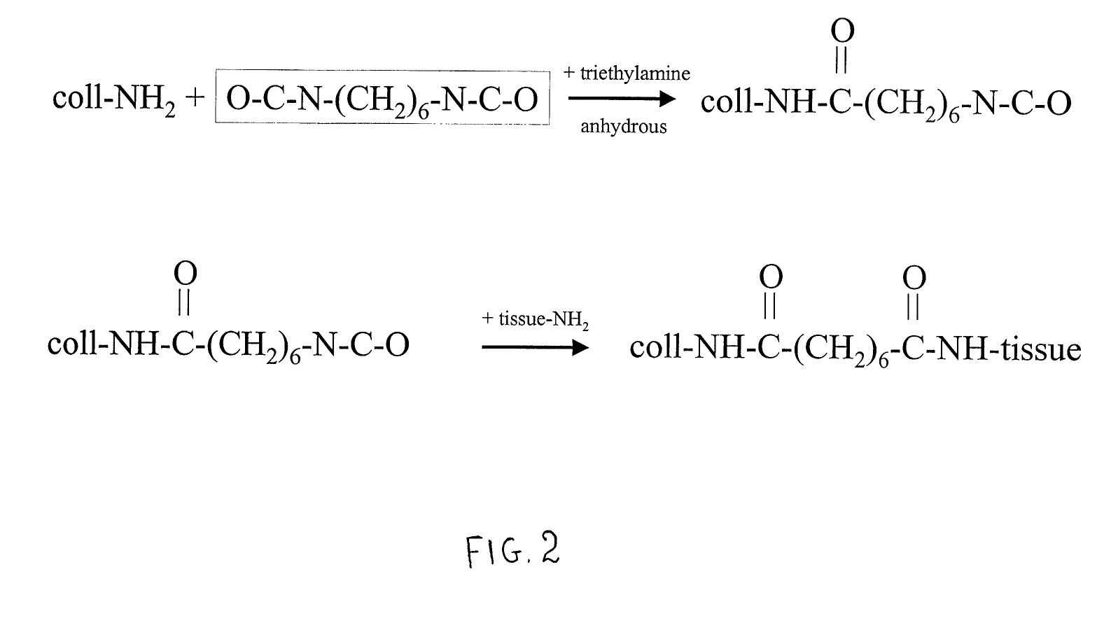

As shown in FIG. 2, in another aspect of the present invention, collagen amine groups (e.g., a (hydroxy)lysine amine group) react with one isocyanate group of a diisocyanate, resulting in the formation of a urea bond. Preferably this reaction is done under anhydrous conditions, as to preserve the pendant isocyanate functionality. It is known that isocyanates are very susceptible to hydrolysis. The yielded product is then stored under essentially dry conditions. Thereafter, when in the presence of water, a crosslink is formed by reaction of the second isocyanate group with a free amine group of the tissue.

Suitable diisocyanates are of the formula

(O)C═N—R—N═C(O)

wherein R is a divalent organic linking group, preferably an aliphatic group optionally substituted with oxygen atoms. More preferably, R is an alkylene group (preferably, having 4-12 carbon atoms) or a polyoxyalkylene group (preferably having an approximate MW of 150-5000).

U.S. Pat. No. 5,817,303 describes the use of protein block copolymers, whereby a polymethylene diisocyanate is used as a crosslinker and to provide the adhesive characteristics. An adhesive according to the present invention can be made in a similar fashion to the method disclosed in Example 3 of U.S. Pat. No. 5,817,303 using a gelatin or collagen solution.

This mechanism of functionalizing a support containing collagen with isocyanate groups, for example, is exemplified in Example 2.

Active Ester Functionalized Collagen Support

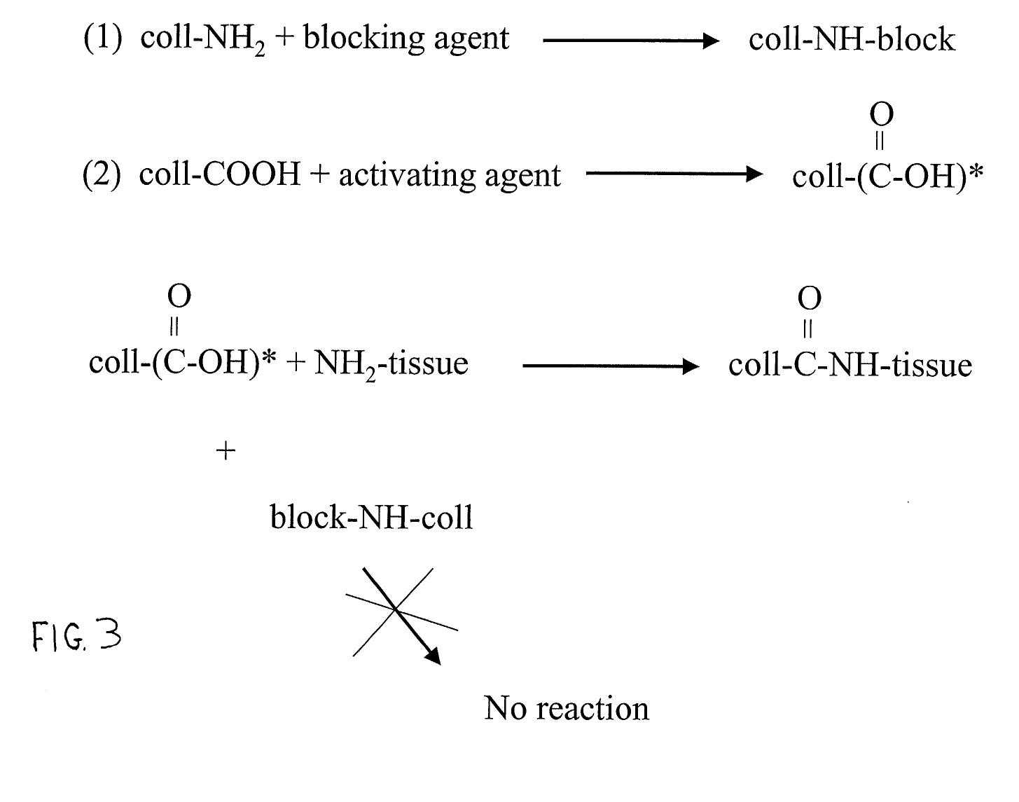

As shown in FIG. 3 , in another aspect of the present invention collagen can be functionalized to yield active esters or activated carboxyl groups capable of subsequent reaction with tissue amino groups to provoke adhesion. Then, first the majority of the pendant (free) amino groups in the collagen support are inactivated by means of blocking, as to prevent reaction with the active esters that are introduced later, leading to internal crosslinking of the collagen support and thus less effective availability of the active ester functionalities to the tissue amino groups. The free amine groups of the collagen support can be blocked by various types of chemical reagents as described in detail below under the section entitled “Activated Ester Adhesive Glue.”

After blocking at least a portion of the amine groups, the complete further functionalization is done under anhydrous conditions. At least a portion of the available carboxyl groups in the collagen support are converted into active esters using a carbodiimide in the presence of NHS, for example, as described in greater detail below. Activation of activating agents such as carbodiimides (e.g., dicyclohexyl carbodiimide (DCC)) gives O-acylisourea groups. In the presence of N-hydroxysuccinimide (NHS) or other suitable stabilizing agents, the O-acylisourea can be converted to an NHS activated carboxylic acid group, that is more stable towards hydrolysis. Thereafter the carbodiimide reagent is removed from the collagen support by rinsing the collagen support. The yielded product is then stored under essentially dry conditions.

Activated Ester Adhesive Glue

In another embodiment, the present invention provides a biological adhesive comprising a stable complex of one or more crosslinkable biomolecules comprising functional groups, which, under aqueous conditions, are capable of bonding to biological tissue. As an example, the free carboxyl groups of biomolecules such as poly(amino acid)s, polysaccharides, and glycosaminoglycans can be functionalized to form activated esters. Examples of poly(amino acid)s include poly(glutamic acid) and poly(aspartic acid). Examples of polysaccharides and glycosaminoglycans include dermatan sulfates, chondroitin sulfates, heparin, heparan sulfates, hyaluronic acid, cyclodextrins, starch, dextrans, dextran sulfates, chitin, and chitosan.

The adhesive glue can be made in a variety of ways. For example, gelatin can be combined with one or more crosslinkable biomolecules having activated ester functional groups and, preferably, amine groups, a portion of which are blocked. Alternatively, the gelatin itself can be chemically modified with activated ester functional groups and to include blocked amine groups.

As an example of an activated ester adhesive glue, in Scheme I shown below, poly(glutamic acid) is initially converted in part into an active ester (e.g., N-hydroxysuccinimide) using a carbodiimide (R₁—N═C═N—R₂), under anhydrous conditions. Activation of the carboxyl groups with activating agents such as carbodiimides (e.g., 1-ethyl-3-(3-dimethyl aminopropyl) carbodiimide-HCl (EDC)) gives O-acylisourea groups. In the presence of N-hydroxysuccinimide (NHS) or other stabilizing agents, the O-acylisourea can be converted to an NHS activated carboxyl group. After the introduction of active esters the obtained intermediate is either kept in an anhydrous solvent, or is dried and stored under essentially dry conditions. When the intermediate is mixed with a solution of gelatin (which includes hydrolyzed collagen), for example, spontaneous gelling occurs as a result of reaction with the free amine groups. When applied to a wound, this gel is believed to adhere to tissue via the formation of covalent bonds.

It is advantageous because substantially no carbodiimide is present in the gel to cause adverse reactions with the biological tissue. That is, although carbodiimide is used in the preparation of the biological adhesive, it can be removed from the intermediate such that it is not a contaminant of the biological adhesive. Added water (or the moisture present in biological tissue) is typically all that is required to cause adhesion of the biological adhesive (and/or adhesive articles on which they are coated or to which they are covalently grafted) to biological tissue.

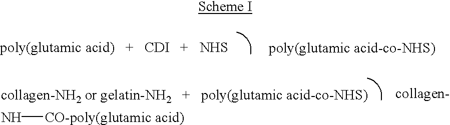

In an alternative embodiment as shown In Scheme II below, the pendant amine groups in gelatin can be inactivated toward amide bond formation, e.g., by acylation. Then at least a portion of the carboxyl groups are converted into an active ester (e.g., N-hydroxysuccinimide) using a carbodiimide (general formula: R₁—N═C═N—R₂), under anhydrous conditions. After the introduction of active esters the obtained intermediate is either kept in an anhydrous solvent, or is dried and stored under essentially dry conditions. The intermediate is then mixed with untreated gelatin in solution, thereby forming an N-hydroxysuccinimide activated gelatin, and applied to the wound. A crosslinked gel will be formed that is believed to adhere to the wound site via covalent bonds. Added water (or the moisture present in biological tissue) is typically all that is required to cause adhesion of the biological adhesive (and/or adhesive articles on which they are coated or to which they are covalently grafted) to biological tissue.

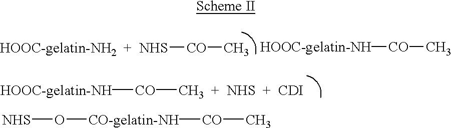

The free amine groups of gelatin can be blocked by various types of chemical reagents if desired. The four major types of reactions through which blocking of free amines can be achieved are: (1) acylation reaction as described above; (2) amination reaction, preferably involving reductive amination using aldehydes or ketones; (3) amination reaction using epoxides; and (4) amination reaction with sulphonyl or sulphonic acid derivatives. Although such reactions involving the use of small blocking agents are preferred, biologically active compounds can also be used to block the free amine groups.

There are numerous acylating agents for use in blocking the amine groups using the acylation reaction. Of particular importance are the isocyanates, isothiocyanates, acid halides, acid anhydrides, activated esters (i.e., those having a good leaving group that is easily released upon reaction with an amine) such as N-hydroxysuccinimide ester, and imidoesters. Preferred acylating agents include, but are not limited to: N-hydroxy succinimide esters (NHS), such as acetic acid N-hydroxysuccinimide ester, sulfo-NHS-acetate, and propionic acid N-hydroxysuccinimide ester; p-nitrophenyl esters such as p-nitrophenyl formate, p-nitrophenyl acetate, and p-nitrophenyl butyrate; 1-acetylimidazole; and citraconic anhydride (reversible blocker).

There are numerous aminating agents (e.g., alkylating agents) for use in blocking the amine groups using the amination reaction. Particularly preferred are aldehydes and ketones. Reaction of a free amine with an aldehyde or ketone yields an imine (or Schiff base) that is quite stable (particularly when an aryl group is present). If necessary, however, the formed imine can be further stabilized through reduction with reducing agents like sodium cyanoborohydride, sodium borohydride, or borane reagents such as dimethylamine borane, trimethylamine borane or morpholine borane.

Aldehydes are preferred aminating agents because ketones generally react more slowly and often require higher temperatures and longer reaction times. A wide variety of aldehydes can be used. Preferably, the aldehydes are monofunctional aldehydes. Examples of monofunctional aldehydes include, but are not limited to, propanal, butanal, hexanal (caproaldehyde), and glyceraldehyde.

Monofunctional epoxides can be also used as the aminating agent to block the amine groups. A monofunctional epoxide forms a secondary amine; however, it is anticipated that such groups will be sufficiently sterically hindered that, under typical reaction conditions, crosslinking will not occur. Suitable monofunctional epoxides include, for example, iso-propylglycidylether and n-butylglycidylether.

Sulphonyl or sulphonic acid derivatives are another group of aminating agents that may be used to block free amine groups. Preferably, the sulphonyl or sulphonic acid derivative is monofunctional. An exemplary reagent is 2,4,6-trinitrobenzenesulfonic acid, for example.

A wide variety of biologically active derivatives of such compounds (i.e., those containing an appropriate reactive moiety such as an ester, aldehyde, or ketone, for example) can be used to block the free amine groups. As a result, desirable biological functions can be included into the collagenous matrix that may improve biocompatibility and overall performance. An example is aldehyde-functional heparin, obtained either through periodate oxidation (periodate-heparin) or nitrous acid degradation (NAD-heparin).

A mixture of the above blocking agents can be used. The blocking agent (or mixture of blocking agents) is used in an amount effective to block at least a portion, preferably, a majority (i.e., greater than about 50%), of the free amine groups. More preferably, the blocking agent(s) is used in a significant molar excess relative to the number of free amine groups.

The blocking reaction is preferably carried out in an aqueous solution, and more preferably, in a buffered aqueous solution having a pH of about 5 to about 8.

Preferably, such blocking agents are capable of blocking at least about 75% of the free amine groups, more preferably, at least about 80%, and most preferably, at least about 90%, of the free amine groups.

Vinylsulfone Adhesive Glue

A preferred embodiment of the present invention includes a biological adhesive comprising a stable complex of one or more crosslinkable biomolecules comprising vinylsulfone functional groups, which, under aqueous conditions, are capable of bonding to biological tissue.

The vinylsulfone adhesive glue can be made in a variety of ways. For example, gelatin can be combined with one or more crosslinkable biomolecules having vinylsulfone functional groups. Alternatively, the gelatin itself can be chemically modified with vinylsulfone functional groups.

In a preferred method, a vinyl sulfone adhesive glue can be made in a similar fashion to the collagen-based bioadhesive composition described in U.S. Pat. No. 5,936,035, in which synthetic, hydrophilic multifunctionally activated polyethylene glycol (PEG) compounds are used. U.S. Pat. No. 5,936,035 particularly describes the utilization of di-functional PEGs whereby the functionalities encompass active esters. These are known to be hydrolytically unstable, and as such the utilization of vinylsulfone compounds is an improvement as the adhesive composition can be premade, not requiring in situ mixing, as is needed with the method of U.S. Pat. No. 5,936,035.

To prepare the collagen-based bioadhesive compositions of this embodiment of the present invention, collagen is crosslinked using a multifunctionally activated synthetic hydrophilic polymer containing vinylsulfone groups. The term “multifunctionally activated” refers to synthetic hydrophilic polymers which have, or have been chemically modified to have, two or more functional groups located at various sites along the polymer chain that are capable of reacting with nucleophilic groups, such as primary amino (——NH₂) groups or thiol (——SH) groups, on other molecules, such as collagen. Each functional group on a multifunctionally activated synthetic hydrophilic polymer molecule is capable of covalently binding with a collagen molecule, thereby effecting crosslinking between the collagen molecules. Types of multifunctionally activated hydrophilic synthetic polymers include difunctionally activated, tetrafunctionally activated, and star-branched polymers.

Multifunctionally activated polyethylene glycols and, in particular, certain difunctionally activated polyethylene glycols, are the preferred synthetic hydrophilic polymers for use in preparing the compositions of this embodiment of the present invention. The term “difunctionally activated” refers to synthetic hydrophilic polymer molecules which have, or have been chemically modified to have, two functional groups capable of reacting with nucleophilic groups on other molecules, such as collagen. The two functional groups on a difunctionally activated synthetic hydrophilic polymer are generally located at opposite ends of the polymer chain. Each functionally activated group on a difunctionally activated synthetic hydrophilic polymer molecule is capable of covalently binding with a collagen molecule, thereby effecting crosslinking between the collagen molecules.

For use in the present invention, molecules of polyethylene glycol (PEG) are chemically modified in order to provide functional groups on two or more sites along the length of the PEG molecule, so that covalent binding can occur between the PEG and reactive groups on the collagen.

In a general method for effecting the attachment of a first surface to a second surface: 1) collagen and a multifunctionally activated synthetic hydrophilic polymer are provided; 2) the collagen and synthetic polymer are mixed together to initiate crosslinking between the collagen and the synthetic polymer; 3) the collagen-synthetic polymer mixture is applied to a first surface before substantial crosslinking has occurred between the collagen and the synthetic polymer; and 4) the first surface is contacted with a second surface to effect adhesion between the first surface and the second surface. At least one of the first and second surfaces is preferably a native tissue surface.

Applications

The biological adhesives and adhesive articles of the present invention can be used in a wide variety of applications, internal as well as external applications. These include tissue adhesion, hemostasis, and sealing of air and body fluid leaks in surgery, as well as on patch electrodes to adhere defibrillation leads. An example of a patch electrode in which the biological adhesive or adhesive article can be used is disclosed in U.S. Pat. No. 5,527,358.

The invention will be further described by reference to the following detailed examples. These examples are offered to further illustrate the various specific and illustrative embodiments and techniques. It should be understood, however, that many variations and modifications may be made while remaining within the scope of the present invention.

### EXPERIMENTAL EXAMPLES

### Example 1

### Vinylsulfone Functionalized Support

Collagen sheets were prepared as follows: 1 gram of collagen was suspended in 200 milliliters (ml) deionized (DI) water using a blender and filtered over a 20 micron×20 micron filter. Heparin was dissolved to achieve 50 milligrams per milliliter (mg/ml) in a 0.05 molar (M) phosphate buffer solution (pH=6.88). To the heparin solution, 3.3 mg/ml NaIO₄ was added, and oxidation of the heparin was allowed to proceed overnight with the exclusion of light.

Just before casting of the collagen, 4 ml of heparin solution and 20 mg NaCNBH₃ were added to 200 ml collagen suspension. Aliquots of 20.4 ml of the obtained mixture were poured into polystyrene weighing boats and allowed to air dry into a solid film. After drying, the obtained films were rinsed with approximately 30 ml DI water, 1 M NaCl, and DI water again. Each step took about 1 hour. The washed sheets were air dried again overnight, followed by drying under vacuum, also overnight, and stored over CaSO₄ before use.

Disks of dry heparin-crosslinked collagen (diameter=14 millimeters), were incubated with NHS-PEG-VS, 1 percent by weight (wt-%) in 2 grams of DMAC, DMSO, or formamide for 30 minutes. The solutions contained 1 wt-% triethylamine as proton scavenger. After modification, the collagen pieces were rinsed three times with THF and dried overnight under vacuum.

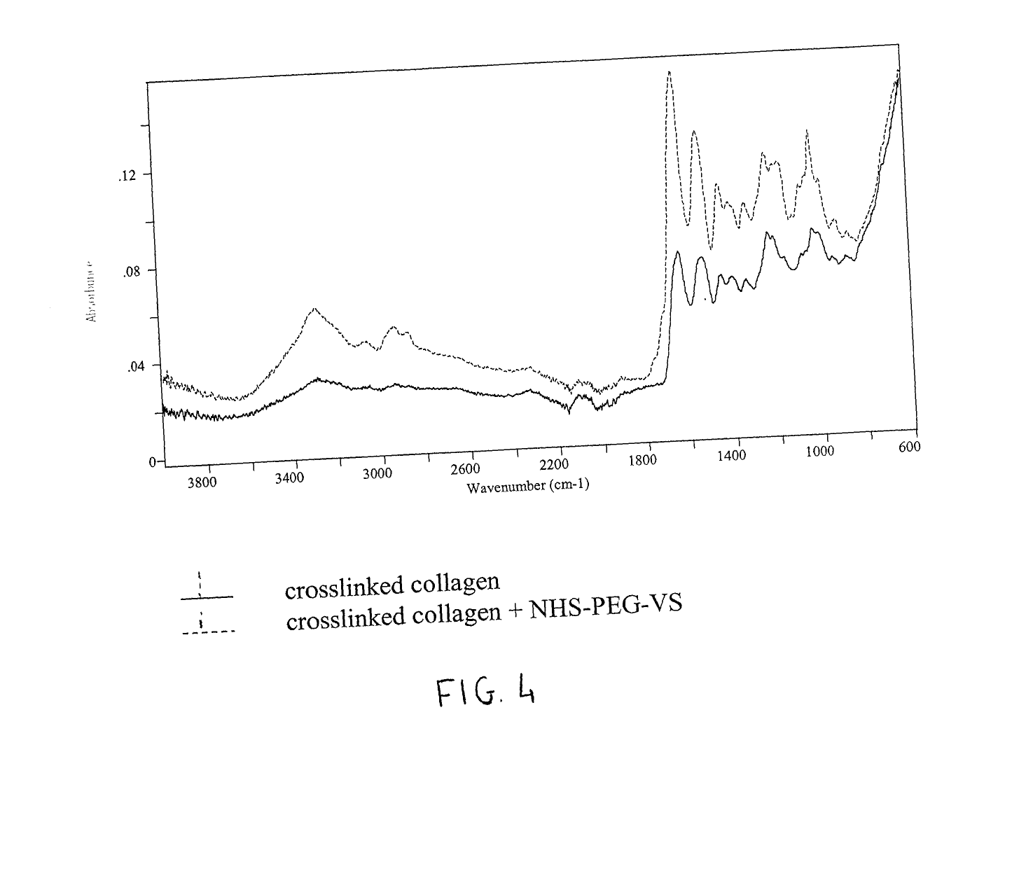

An IR spectrum, as shown in FIG. 4, did show attachment of the PEG molecule to the collagen, as was concluded from the occurrence of an absorbance band at 2900 cm⁻¹, when formamide or DMSO was used as solvent. Exposure of the treated collagen to a polyamine molecule, polyethyleneimine in this case, showed (qualitatively) more amines present using TNBS staining. Thus, principally this approach seems feasible.

### Example 2

### Isocyanate Functionalized Support

Collagen sheets were prepared as follows: 1 gram of collagen was suspended in 200 ml DI water using a blender and filtered over a 20 micron×20 micron filter. Heparin was dissolved to achieve 50 mg/ml in a 0.05 M phosphate buffer (pH=6.88). To the heparin solution, 3.3 mg/ml NaIO₄ was added, and oxidation of the heparin was allowed to proceed overnight with the exclusion of light.

Just before casting of the collagen, 4 ml of heparin solution and 20 mg NaCNBH₃ were added to 200 ml collagen suspension; aliquots of 20.4 ml of the obtained mixture were poured into polystyrene weighing boats and allowed to air dry into a solid film. After drying, the obtained films were rinsed with approximately 30 ml DI water, 1 M NaCl and DI water again. Each step took about 1 hour. The washed sheets were air dried again overnight, followed by drying under vacuum, also overnight, and stored over CaSO₄ before use.

Pieces of dry heparin-crosslinked collagen sheets, 4 cm×4 cm (centimeter), were incubated with 5 ml solutions of 1 volume % or 10 volume % (v/v) hexamethylene diisocyanate (HMDI) in THF and formamide, or combinations thereof, and DMSO containing 0 or 1 wt-% triethylamine as proton scavenger, for approximately 1 hour. After incubation all sheets were rinsed twice with THF and were dried under vacuum overnight. Samples were then stored over CaSO₄.

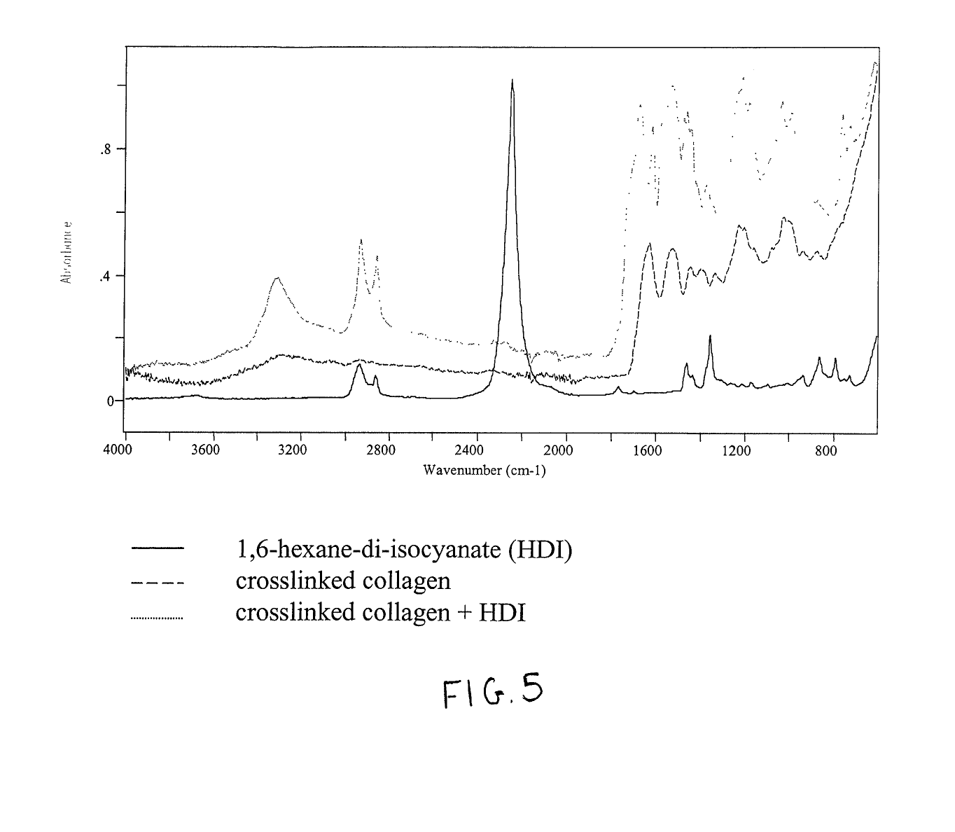

The IR spectrum of the collagen sheets treated with 10% HMDI in THF, containing 1% triethyleamine, as shown in FIG. 5, did not show the expected absorbance at 2200 and 1690cm⁻¹ for —N═C═O. Some absorbance was observed at 3000 cm⁻¹ and 3400 cm⁻¹, that may have been caused by the introduction of —(CH₂)₆— structures and the presence of extra —NH₂ groups (as compared to non-treated collagen sheet).

Collagen sheets treated with 10% HMDI in formamide, or formamide mixed with THF (75/25, 50/50 and 25/75) did show swelling, especially with the higher formamide concentrations. The IR spectrum showed stronger bands at 3000 cm⁻¹, due to the presence of —(CH₂)₆— and 3400 cm⁻¹, indicative of extra —NH₂, and also a band at 1750 cm⁻¹, indicative of urethane and urea urethane structures (formed upon reaction of isocyanate with amine group).

Omitting triethylamine from the activation solution did not give the modifications of the IR spectrum as seen above. This is because protonation of the lysinyl amines, and hence slow down of the addition to the isocyanate group.

Treating a collagen sheet with HMDI in DMSO gave results comparable to what was observed with formamide; the presence of urethane and urea urethane groups, extra primary amines and methylene groups.

Measurable attachment of HMDI to the collagen sheet can be best achieved in a solvent that induces swelling of the collagen sheet, such as observed with formamide or DMSO, in the presence of triethylamine. However, hydrolysis of the pendant —C═N═O groups in to —C—NH₂ groups seems to occur rapidly. Thus, more careful exclusion of water appears to be necessary.

### Example 3

### Vinylsulfone Functionalized Support

Collagen sheets were prepared as follows: the collagen (type I) used to prepare the collagen sheets was supplied by Sigma and is made from bovine achilles tendon. Collagen (1 gram) was suspended in 200 ml 0.3 wt-% acetic acid with a blender and filtered over a 20 micron filter. Twenty grams of the obtained suspension was poured in polystyrene weight boats and allowed to air dry into a solid film.

Collagen sheets crosslinked with periodate oxidized heparin (crosslinked collagen sheets) were prepared as follows: 1 gram of collagen was suspended in 200 ml 0.3 wt-% acetic acid with a blender and filtered over a 20 micron filter. Heparin was dissolved up to 50 mg/ml in a 0.0025 M Na₂PO₄ buffer (pH=6.8). NaOl₄ was added to achieve a final concentration of 3.3 mg/ml and oxidation was allowed to proceed overnight. Just before casting, 4 ml of the heparin solution and 20 mg NaCNBH₃ were added to 200 ml of the collagen suspension. A portion of the mixture (20.4 g) was poured in polystyrene weight boats and allowed to dry into a solid film.

After drying, the sheets were rinsed with DI water, 0.9 wt-% NaCl, and again with DI water. Each washing step was carried out for 30 minutes. The rinsed sheets were then air dried again overnight.

A collagen sheet and a crosslinked collagen sheet were incubated with a solution of NHS-PEG-VS in DMSO (0.8 % wt/wt) for 3 hours. The solution contained 0.8 wt-% triethylamine as a proton catcher. The sheets were rinsed 3 times with THF and dried overnight under vacuum.

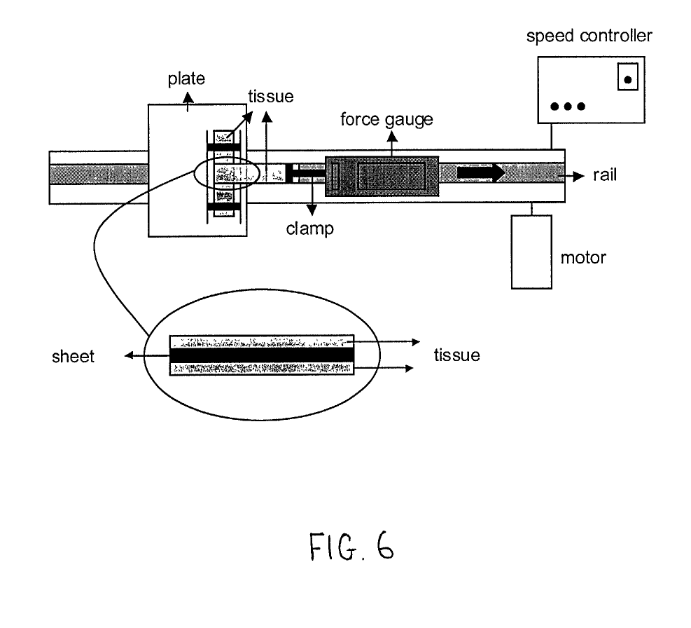

Adhesion or bonding strength between a collagen sheet and two pieces of porcine heart tissue was measured with the method illustrated in FIG. 6. Fresh porcine heart tissue obtained from a local slaughterhouse was stored at 4° C. until use. A piece of the porcine heart was removed and cut into 7 cm×2 cm pieces. The thickness of the tissue was approximately 5 mm. The adhesion between different collagen sheets and porcine heart tissue was determined as follows: a dry piece of the collagen sheet was placed on the innerside of the porcine heart tissue, after applying a few droplets of DI water, the other piece of porcine heart tissue of the same size was put on it to have a bonding area of 1.5 cm×1.5 cm. After applying a load of 80 g for 10 minutes, bonding strength was measured using a force gauge.

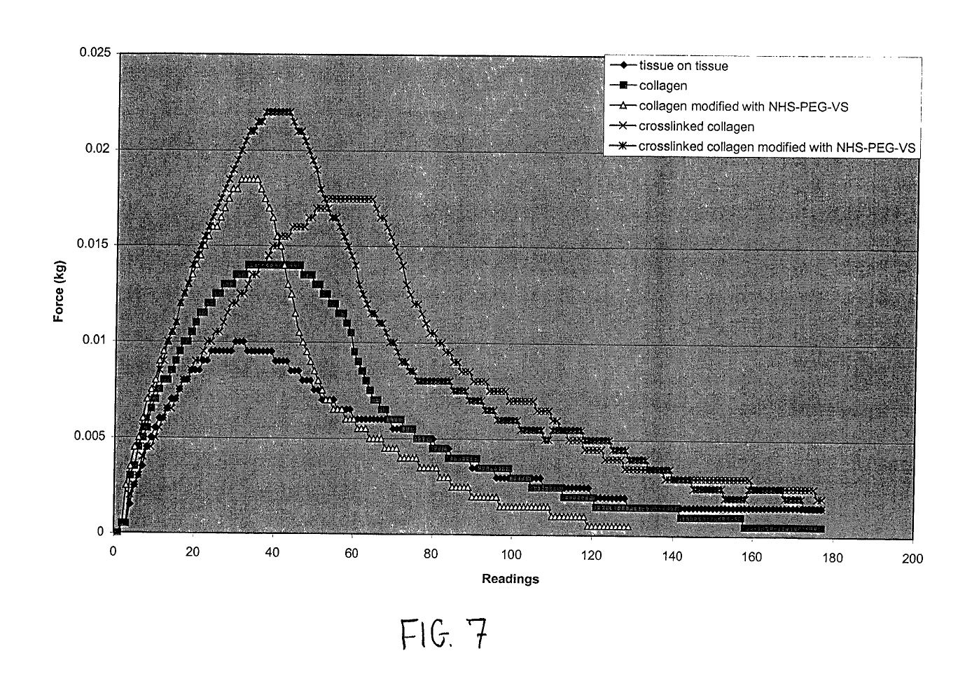

The bonding strength of the collagen sheets and the crosslinked collagen sheets with and without the vinylsulfone functionality, as introduced through reaction of the NHS group with free amines within the collagen material, is illustrated in FIG. 7. The figure shows that modification of the collagen sheets by reaction with NHS-PEG-VS gives an increase in bonding strength.

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A biological adhesive article comprising a solid support comprising covalently bound functional groups pendant therefrom, wherein the functional groups are selected such that they are reactive with biological tissue when in an aqueous environment to cause adhesion of the support to the tissue.

2. The adhesive article of claim 1 wherein the solid support is substantially insoluble in water.

3. The adhesive article of claim 2 wherein the water-insoluble support comprises collagen.

4. The adhesive article of claim 3 wherein the collagen is reconstituted and purified collagen.

5. The adhesive article of claim 3 wherein at least a portion of the collagen is crosslinked.

6. The adhesive article of claim 1 wherein the functional groups are selected from the group of isocyanates, vinylsulfones, activated esters, and mixtures thereof.

7. The adhesive article of claim 6 wherein the functional groups are selected from the group of isocyanates, vinylsulfones, and mixtures thereof.

8. The adhesive article of claim 7 wherein the functional groups are vinylsulfones.

9. The adhesive article of claim 1 which is in the form of a patch electrode.

10. The adhesive article of claim 1 wherein the solid support comprises collagen and the functional groups are selected from the group of isocyanates, vinylsulfones, activated esters, and mixtures thereof.

11. The adhesive article of claim 1 further comprising one or more bioactive molecules.

12. The adhesive article of claim 11 wherein the bioactive molecule is selected from the group of an angiogenic factor, a growth factor, an antimicrobial agent, an antithrombotic agent, an anticalcification agent, an anti-inflammatory agent, an anti-arrhythmic agent, an analgesic and combinations thereof.

13. The adhesive article of claim 1 wherein the functional groups form covalent bonds with the biological tissue when in an aqueous environment.

14. The adhesive article of claim 13 wherein the functional groups form covalent bonds with amine and/or thiol groups of the biological tissue.

15. The adhesive article of claim 1 wherein the solid support further comprises free amine groups, a portion of which are blocked.

16. A biological adhesive article comprising a solid support comprising collagen and covalently bound functional groups pendant therefrom, wherein the functional groups are selected such that they are reactive with biological tissue when in an aqueous environment to cause adhesion of the support to the tissue through the formation of covalent bonds, and further wherein the functional groups are selected from the group of isocyanates, vinylsulfones, activated esters, and mixtures thereof.

17. A method of attaching an article to biological tissue, the method comprising:
providing a biological adhesive article comprising a solid support comprising covalently bound functional groups pendant therefrom, wherein the functional groups are selected such that they are reactive with biological tissue when in an aqueous environment; and
contacting the biological adhesive article to the biological tissue to cause adhesion of the support to the tissue.

18. The method of claim 17 wherein adhesion occurs through the formation of covalent bonds between the functional groups and the biological tissue.

19. The method of claim 18 wherein the article is a self-sticking pad.

20. The method of claim 17 wherein the biological adhesive article is contacted with water prior to contacting it to the biological tissue.

21. The method of claim 17 wherein the solid support comprises collagen.

22. A method of preparing a self-sticking pad, the method comprising:
providing a solid support; and
functionalizing the solid support with covalently bound functional groups pendant therefrom, wherein the functional groups are selected such that they are reactive with biological tissue when in an aqueous environment to cause adhesion of the support to the tissue.

23. The method of claim 22 wherein the solid support comprises collagen.

24. The method of claim 23 wherein the functional groups are selected from the group of isocyanates, vinylsulfones, activated esters, and mixtures thereof.

25. The method of claim 22 wherein the functional groups are provided by chemically modifying the solid support to form pendant functional groups directly bonded to the solid support.

26. A biological adhesive comprising a stable complex of one or more crosslinkable biomolecules comprising vinylsulfone functional groups.

27. The adhesive of claim 26 comprising gelatin.

28. The adhesive of claim 26 which is coated on a solid support.

29. A method of sealing a wound, the method comprising contacting the wound with a biological adhesive comprising a stable complex of one or more crosslinkable biomolecules comprising vinylsulfone functional groups.

30. A method of forming a biological adhesive, the method comprising combining gelatin with one or more crosslinkable biomolecules comprising vinylsulfone functional groups.

31. A method of forming a biological adhesive, the method comprising chemically modifying gelatin with vinylsulfone functional groups.

32. A biological adhesive comprising a stable complex of one or more crosslinkable biomolecules comprising free amine groups, a portion of which are blocked, and functional groups, which, under aqueous conditions, are capable of bonding to biological tissue.

33. A method of sealing a wound, the method comprising contacting the wound with a biological adhesive comprising a stable complex of one or more crosslinkable biomolecules comprising free amine groups, a portion of which are blocked, and functional groups, which, under aqueous conditions, are capable of bonding to biological tissue.

34. A method of forming a biological adhesive, the method comprising combining gelatin with one or more crosslinkable biomolecules comprising free amine groups, a portion of which are blocked, and functional groups, which, under aqueous conditions, are capable of bonding to biological tissue.

35. A method of forming a biological adhesive, the method comprising chemically modifying gelatin with fundtional groups, which, under aqueous conditions, are capable of bonding to biological tissue, wherein the gelatin comprises free amine groups, a portion of which are blocked.
